(19) United States
(12) Patent Application Publication
(10) Pub. No.: US 2010/0317967 A1
(43) Pub. Date: Dec. 16, 2010

(54) COMPUTER ASSISTED THERAPY MONITORING

(75) Inventors: Ingwer-Curt Carlsen, Hamburg (DE); Stewart M. Young, Hamburg (DE); Kirsten R. Meetz, Hamburg (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/521,601

(22) PCT Filed: Dec. 5, 2007
(86) PCT No.: PCT/IB07/54935

§ 371 Date: Jun. 29, 2009

Related U.S. Application Data
(60) Provisional application No. 60/883,180, filed on Jan. 3, 2007.

(51) Int. Cl. A61B 5/05

(52) U.S. Cl. 600/427

(57) ABSTRACT

A computer assisted therapy apparatus (100) uses data from functional medical imaging examinations of a patient to evaluate the response of a patient to an applied therapy. A lesion tracker (112) tracks lesion(s) detected in the medical imaging examinations, and a lesion quantifier (114) generates quantitative information indicative of functional characteristic of the lesion(s). A trend analyzer (116) uses the quantitative information to determine trends in the functional characteristic.

The present application relates to computer assisted therapy in medicine. It finds particular application to the use of functional image data in therapy, for example in connection with the use of information from nuclear medicine (NM) and computed tomography (CT) examinations in oncology.

Recent years have seen the development of computer assisted diagnosis (CAD) systems. These systems support radiologists and other medical professionals by analyzing medical image data to identify suspicious lesions. In x-ray mammography, for example, CAD systems have been used to aid in the identification of breast tumors. In CT applications, CAD systems have been used in connection with the identification and classification of lung cancer. Indeed, the development of CAD systems, together with advancements in functional imaging modalities such as single photon emission computed tomography (SPECT), positron emission tomography (PET), and multi-modality imaging systems such as PET/CT and SPECT/CT systems, have significantly improved the ability to detect and identify cancer.

Of course, successful clinical outcomes depend on an effective therapy. Traditionally, therapy response assessment has used morphological criteria to assess the response to a therapy. In one such technique, the effectiveness of a therapy has been gauged by using images taken before and after a course of treatment to determine the change in size of a tumor. As will be appreciated, however, these morphological techniques have provided relatively limited and untimely information about the characteristics of the tumor or the effectiveness of the therapy.

Advances in PET imaging techniques, together with the development of tracers such as FDG, FLT, and FMISO, have led to an increasing interest in PET imaging as a therapy assessment tool. Depending on factors such as the particular imaging modality, tracer, and disease type, data from an imaging examination can be used to gauge the health or aggressiveness of a tumor, its expected sensitivity to applied radiation or other therapy, and the like. This information can in many cases be used not only to tailor an initial therapy, but also to gauge the effectiveness of an applied therapy, or to adjust or further tailor the applied therapy, for example by adjusting an applied radiation or other therapy dose, introducing a different or adjunct therapy, or redirecting the patient to palliative care. Moreover, the information can often be obtained relatively earlier in the therapeutic process, thereby enhancing the likelihood of a successful outcome and potentially reducing the use of ineffective or ultimately unnecessary treatments.

In current clinical practice, a baseline FDG-PET/CT scan is often obtained prior to treatment. Follow-up scans are taken during the course of therapy, for example after one or more cycles of a chemotherapy regimen. The physician has manually identified lesions and other regions of interest (ROIs) in the image data, and changes in the standardized uptake values (SUVs) of the identified lesions have been used to assess the therapy response.

While such an approach represents an improvement over traditional morphological assessment techniques, there nonetheless remains room for improvement. For example, the manual identification and delineation of lesions is often a relatively labor intensive, subjective task which is subject to inter- and even intra-physician variation. As another example, the functional or metabolic information derived from an imaging examination can vary as function of differences in imaging protocol and patient population. In the case of a particular patient, variations in equipment availability, patient preparation time, metabolic state, and the like may influence the functional imaging data and hence the conclusions drawn from data acquired at different times during the course of a therapy. Other variations in the applied imaging protocol (e.g., differences in imaging times, imager settings, and/or tracer administration) can likewise lead not only to intra- and inter-patient variations, but also to variations between various physicians and institutions. Moreover, the functional data and its assessment may vary due to disease and tracer-specific factors.

Thus, in the case of a time series of images of a particular patient, these variations can make it more difficult to assess the metabolic state of a tumor and the response to a particular therapy. More generally, these variations also complicate the development of objective therapy and therapy response assessment criteria across the broader patient population.

Aspects of the present application address these matters and others.

In accordance with one aspect, an apparatus includes a lesion detector which detects lesions in medial image data from an imaging examination of a patient, a lesion quantifier in operative communication with the lesion detector, and a trend analyzer. The lesion quantifier uses first functional image data from a first imaging examination of the patient conducted before the application of a therapy to the patient to generate first lesion functional data for a first detected lesion and second functional image data from a second imaging examination of the patient conducted after the application of the therapy to generate second lesion functional data for the first detected lesion. The trend analyzer identifies a difference between the first and second lesion functional data.

According to another aspect, a method includes detecting a lesion in first medical image data from a first imaging examination of a patient, detecting the lesion in second medical image data from a second imaging examination of the patient conducted after the application of a therapy to the patient, using data from the first imaging examination to generate first functional data indicative of a functional characteristic of the lesion, using data from the second imaging examination to generate second functional data indicative of the functional characteristic, calibrating the first functional data to generate first calibrated functional data, calibrating the second functional data to generate second calibrated functional data, and using the first and second calibrated functional data to evaluate a response of the lesion to the therapy.

According to another aspect, a computer readable storage medium contains instructions which, when executed out by a computer, cause the computer to carry out a method. The method includes using medical image data from a first functional medical imaging examination of a patient to generate a first lesion functional data for a lesion present in the anatomy of the patient. The method also includes using the first lesion functional data and a second lesion functional data for the lesion obtained from a second functional medical imaging examination of the patient to evaluate the response of the lesion to an applied therapy.

According to another aspect, a computer readable storage medium contains a data structure. The data structure includes a first motion model and a first physiology model. The motion model contains data which, when accessed by a lesion tracker, describes an expected motion of a lesion detected in data from a medical imaging examination of a patient. The physiology model contains data which, when accessed by a lesion quantifier, describes an expected behavior of a first tracer applied to the patient in connection with a functional medical imaging examination.

According to another aspect, a method includes receiving a physiology model which, when accessed by a lesion quantifier, models at least one of an expected behavior of an imaging agent and a physiological characteristic of a patient in connection with a functional medical imaging examination and storing the computer readable data in a computer readable memory accessible to the lesion quantifier.

According to another aspect, a method for use in computer assisted therapy monitoring includes using information which describes an image protocol used in connection with a functional imaging examination of a patient to select model data and using the selected model data to vary an operation of a component of a computer assisted therapy monitoring system.

Still further aspects of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

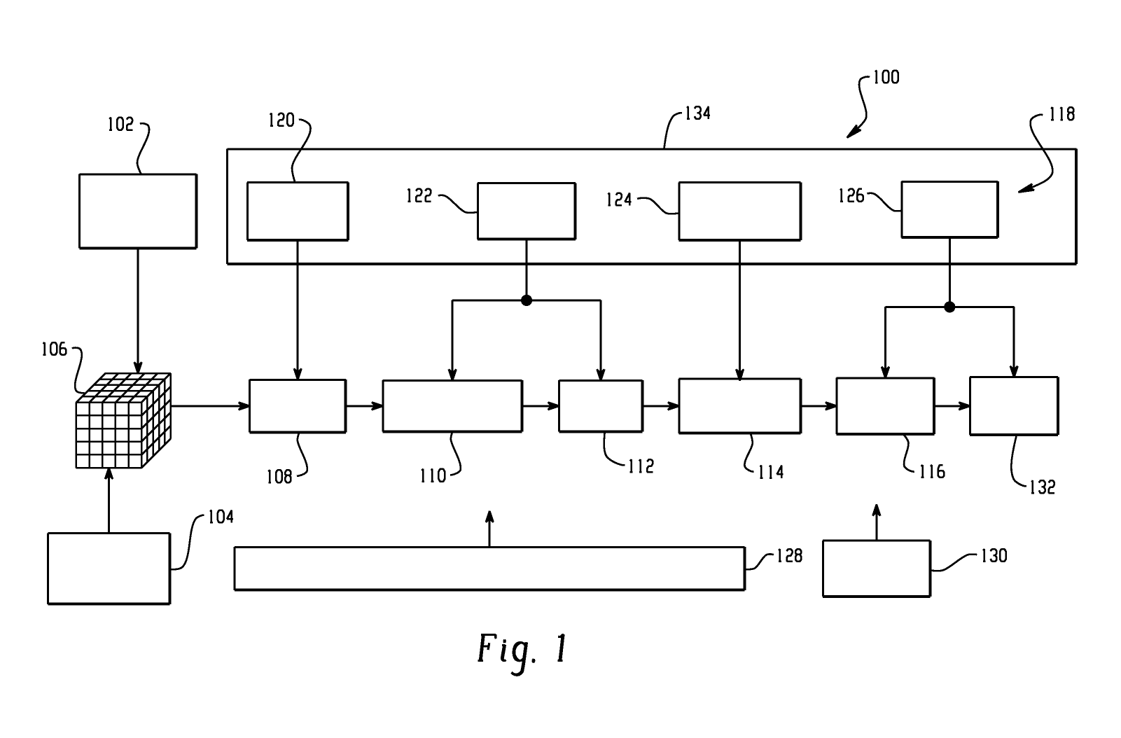

FIG. 1 depicts a computer assisted therapy apparatus.

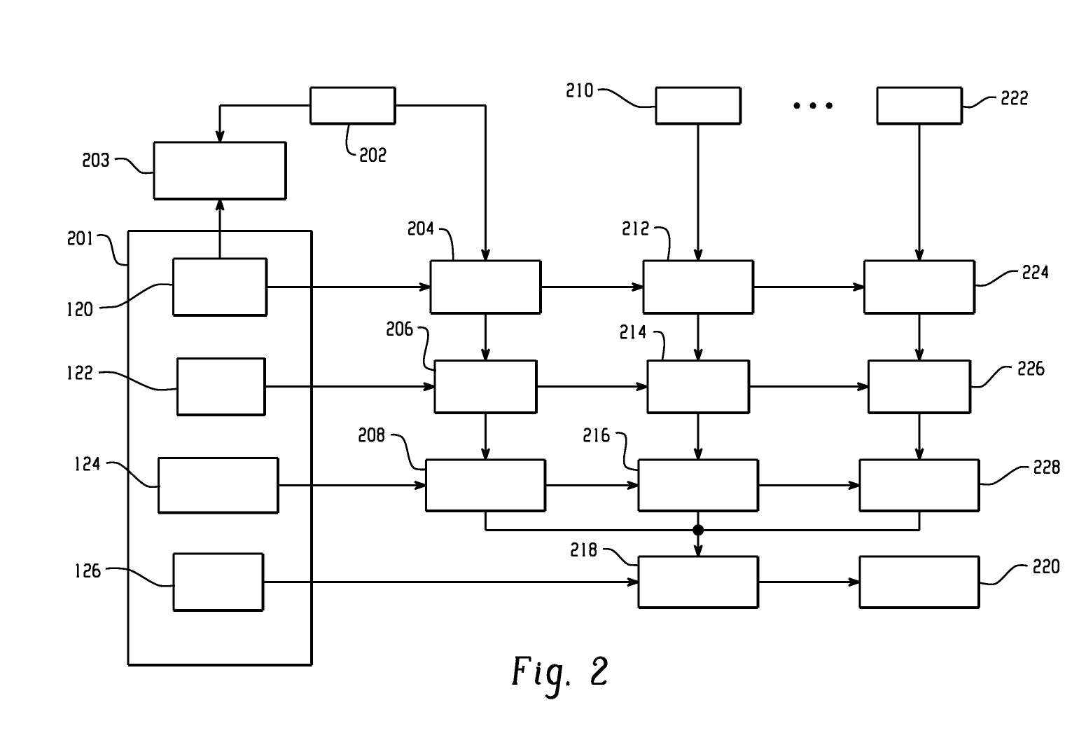

FIG. 2 depicts a computer assisted therapy method.

With reference to FIG. 1, a computer assisted therapy monitoring system 100 includes a functional medical imager 102 and a structural medical imager 104 which generate volumetric image data 106 indicative of a subject patient or other object under examination. The functional medical imager 102 provides functional or metabolic information, while the structural imager 104 provides information indicative of the object's structure or morphology. Exemplary functional imaging modalities include PET, SPECT, functional magnetic resonance imaging (fMRI), and molecular imaging. PET and SPECT systems measure the decay of radionuclides introduced into the anatomy of a patient. Depending on the tracer used, such examinations can be used to provide information indicative of functional characteristics such as metabolism in the case of FDG, cell proliferation in the case of FLT, and hypoxia in the case of FMISO.

Examples of structural imaging modalities include CT, MRI, x-ray, and ultrasound (US). Though illustrated as separate systems, it will be appreciated that the functional 102 and structural 104 imagers may be combined in a single system, for example as a PET/CT, SPECT/CT, PET/MR, or other such scanner. Of course, the above examples are non-limiting; a single modality may serve as both a structural and functional imager.

The system 100 also includes image processing components such as a lesion detector 108, a registration processor 110, a lesion tracker 112, a lesion quantifier 114, and a trend analyzer 116. The image processing components are advantageously implemented via computer readable instructions which, when carried out by a computer processor(s), cause the computer(s) to perform the functions of the respective components. Model data 118 (which includes one or more of anatomic model(s) 120, motion model(s) 122, physiology model(s) 124, and disease model(s) 126) and patient specific data 128 are stored in a computer readable memory or memories which are part of or otherwise accessible to the various components.

The model data 118 is advantageously maintained in a modular data structure (or structures) distinct from the executable code of the various image processing components. In one such implementation, the model 118 and patient specific 128 data are stored in a hospital information system/radiology information system (HIS/RIS) system and accessed via a suitable communications network. In another, some or all of the data 118, 128 is maintained in memory associated with the computer or computers of the system 100. In still another, some or all of the data is stored in database maintained at a remote location and accessed via a wide area network (WAN) or other suitable communications network. Depending on the requirements of a particular application, such a data structure may be exploited to facilitate the use of common image processing components with different model data 118, the consistent application of the model data 118 to a particular patient or across multiple patients, physicians or institutions, and/or the implementation of upgrades, updates, or other changes to the model data 118.

An operator interface 130 including a display or other output device(s) and input devices such as a mouse and/or keyboard allow the user to control the operation of or otherwise interact with the various components of the system 100 using a graphical user interface (GUI) or other suitable interface.

The lesion detector 108, which is advantageously implemented as a CAD system, analyzes the image data 106 to identify candidate lesions, for example based on a combined analysis of the available morphological and functional image data. The lesion detector 108 operates in conjunction with the anatomical model(s) 120, which provide a priori information indicative of the structures or features to be identified. Exemplary anatomical models include surface-based representations of organ boundaries and volume-based or volumetric representations of the three-dimensional anatomy.

The lesion detector 108 also operates in conjunction with ancillary information which links the anatomical model data 120 to the patient specific data 128. Examples of such ancillary information include anatomical landmarks, global geometric relations, and the like. It will also be appreciated that, due to the differences in characteristics of the various imaging modalities, the anatomical model(s) 120 and ancillary data may vary based on the modalities selected for the functional 102 and structural 104 imagers.

Note that the consistent application of the model data 118 and other information can ordinarily be expected to improve the consistency of the lesion detection and delineation for a particular patient or across multiple patients. Nonetheless, it may be desirable to present candidate lesions to the clinician via the operator interface 130, with the clinician being afforded the opportunity to accept or reject one or more of the lesions, adjust their delineation, or the like. The clinician may also be afforded the opportunity to manually identify still other lesions. In another implementation, the lesion detection is performed automatically without operator intervention. In either case, the identified lesion(s) are tagged or otherwise identified, and the information is stored in a suitable computer readable memory for further use.

The registration processor 110 registers the coordinate systems of the image data 106 to account for misalignments between the various images. For example, the registration processor 110 reconciles the coordinate systems of the image data 106 from the functional 102 and morphological 104 imagers to account for gross and/or periodic patient motion during the course of a given scan. In the case of image data 106 acquired at various times over the course of a treatment regimen, the registration processor 110 reconciles the coordinate systems of the time series of images.

Exemplary registration techniques include both surface and volume-based techniques. Surface-based registration generally uses organ, lesion, or other boundaries (e.g., as identified by the lesion detector 108) to align the coordinate systems. Surface-based registration is particularly well-suited for use in radiation therapy applications, as the planning of an administration of radiation therapy has traditionally used organ or lesion surface contours derived from CT images. Volume-registration techniques, on the other hand, typically eschew an explicit segmentation operation and operate instead on the volumetric data.

The registration processor 110 operates in conjunction with the motion model(s) 122, which can be used to provide a priori or other information about expected motion, for example due to respiratory motion, cardiac motion, or differences in the filling of the bladder or rectum. It is generally desirable to provide an appropriate mathematical description of the expected motion patterns, determine reasonable initial values for describing a misalignment, and to guide the optimization of these parameters to achieve a desired alignment. Exemplary motion models 120 include one or more alternative mathematical transformations which can be selected based on an expected motion pattern, a vector field giving a typical motion over a field of view (FOV) or region of interest, (ROI) or at anatomical landmarks, or models which can be directly integrated into the anatomical model(s) 120 in the form of dynamic surface models or otherwise. While the consistent application of the model data 118 and other information can again be expected to improve the consistency of the registration process, the registration may be performed in a semi-automatic or automatic fashion analogous to that of the lesion detection.

The lesion tracker 112, which likewise operates in conjunction with the motion model(s) 122, tracks one or more candidate lesions over the course of a time series of images. In this regard, it should be noted that the lesion tracking may also be accomplished in the absence of a complete image registration. For example, the lesion tracker 112 may operate in conjunction with one or both of the anatomical 120 and motion 122 models to determine the correspondence of lesion(s) detected in one or more of the time series of images. The lesion tracker 112 may likewise operate in a semi-automatic or automatic fashion. In a semi-automatic implementation, for example, the user may be afforded the opportunity to accept or reject a proposed correspondence between lesions detected in various images of a time series of images, define new or different correspondences, or the like. Again, the lesion tracker 112 stores the correspondence between the various lesions in a suitable data structure in a memory which is part of or otherwise accessible to the system 100.

The lesion quantifier 114 provides quantitative information the various tracked lesions. More particularly, it is desirable that the image data be calibrated or normalized to arrive at quantitatively correct and reproducible lesion functional data. Hence, the lesion quantifier 114 operates in conjunction with the patient specific data 128, for example using patient-specific anatomical morphology to compensate for partial volume effects which can result from the differing spatial resolutions of the functional 102 and structural 104 imagers. The lesion quantifier also operates in conjunction with the physiology model(s) 124 to account for the dynamic behavior of the tracer or physiological variations between patients. Thus, the physiology models(s) include information such as one or more of the expected tracer uptake locations, uptake and washout times, physiological relationships, or other information which models the expected behavior of the particular tracer in connection with the physiology of interest.

The lesion quantifier 114 advantageously uses this information to account for variations resulting from factors such as one or more of differences in imaging protocols (e.g., in patient preparation, tracer doses, or in imager settings), differences in patient physiology, and the like. The functional data may be calibrated or normalized to reduce the effects of inter-institution, inter-physician, inter-patient, intra-patient, or other variations. One example of lesion functional data generated by the lesion quantifier 114 which is particularly useful in connection with tracers such as FDG includes normalized standard uptake values (SUVs) for the various lesions. Other functional indicators include cell proliferation, hypoxia, or other functional indicators, it being understood that the functional indicators are typically a function of the applied tracer.

The physiology model(s) 124 can be provided in various forms. In one implementation, the physiology model information 124 is provided via analytical expressions (i.e., pharmacokinetic models) which describe exchange of the tracer between different anatomical or physiological compartments. Depending on factors such as the particular pathology and tracer, the therapy to be delivered, and the desired accuracy, the parameters and values may be derived from pharmaceutical databases. The model data may also be empirically derived based on the observed responses of patients at a particular institution, of particular patient classes or cohorts, of individual patients model, or based on observed variations resulting form the use of different models or types of imagers 102, 104, imagers manufactured by different vendors, or the like.

The trend analyzer 116 evaluates the normalized lesion functional data generated by the lesion quantifier 114 to generate therapy response indicators across one or more points in the time series of image acquisitions and hence assess the response to the applied therapy. In addition to functional response indicators, the trend analyzer my also consider morphological response indicators such as the size, shape, boundaries or other morphological characteristics of the lesions as determined using information from the structural imager 104. To this end, the trend analyzer 126 operates in conjunction with the disease model(s) 126 and the patient specific data 128 to account for pathology and/or patient specific factors.

In one implementation which is again particularly well suited to FDG-based imaging techniques, the trend analyzer 116 therapy response indicator includes a threshold-based criteria for evaluating normalized SUVs (or changes in the normalized SUVs) generated by the lesion quantifier 114. Still other analyses are also contemplated, for example based on analytical, statistical, or heuristic assessments of the temporal development of desired functional or morphological response indicators. As will be appreciated, the various assessments and the relevant criteria are provided by the disease model(s) 126. Note also that non-image based data (e.g., patient demographic information, the results of chemical assays, or other study information) may also be included in the therapy response assessment process.

A therapy system 132, which again operates in conjunction with the disease model(s) 126 and the patient specific data 128, uses the response assessment(s) provided by the trend analyzer to determine or otherwise suggest a particular therapy. As noted above, for example, the therapy system 132 may suggest an adjustment to the therapy, a different or additional course of therapy, or diversion to palliative care. The therapy or therapy adjustment may also be presented with a confidence level or other information indicative of an expected success or outcome of the therapy so that clinician can use the information in selecting among various alternatives. Exemplary therapies, which are typically pathology and patient-specific, include externally applied radiation therapy, chemotherapy, radio frequency (RF) or other ablation, brachytherapy, surgery, and molecular therapies. The therapy system 132 advantageously operates on a semi-automatic basis so that the clinician is afforded the opportunity to accept or adjust the treatment, although automatic implementations are also contemplated.

Also as illustrated, a knowledge maintenance engine 134 may be used to select the appropriate model data 118 or otherwise implement rules which govern the operation of the various system components based on application specific criteria. Thus, one or more of the anatomic model(s) 120, motion model(s) 122, physiology model(s) 124, or the disease model(s) 126 may include multiple parameter values which are selected based on a particular patient, tracer, imaging protocol, disease, or the like. Similarly, the knowledge maintenance engine 134 may be used to select among more than one possible algorithm for use by the various image processing components. For example, and as noted above, the lesion detector 108 may utilize different parameter values or detection algorithms depending on the modalities of the functional 102 and structural 104 imagers.

Again, the consistent application of the model data 118 and system configuration rules can ordinarily be expected to improve of the therapy response assessment. In any case, the user may be presented with a menu of configuration options or otherwise afforded the opportunity to influence the system configuration, with the knowledge maintenance engine 134 checking to ensure the validity and/or consistency of the various selections based on the appropriate application specific criteria (e.g., in processing images from a PET scan using FDG the knowledge maintenance engine would be used to ensure that FDG-appropriate anatomic 120, motion 122, physiology 124, or disease 126 models are used). In a semiautomatic implementation, the knowledge maintenance engine 134 uses the application specific criteria to propose appropriate models 118 for acceptance by the user. In a fully automatic implementation, the configuration is performed automatically.

Operation of the system 100 will now be described in relation to FIG. 2 for the exemplary case of an FDG PET/CT examination in connection with the treatment of lung cancer. At 200, a baseline scan is obtained prior to treatment. The baseline scan includes a diagnostic quality CT scan which provides patient-specific morphological information and a PET scan which provides functional data as indicated generally at 203. The information 203 may also include patient specific physiological information indicative of metabolic rate or other factors which may influence the lesion functional data.

At 201, the knowledge maintenance engine 134 is used to configure the system, for example to select the appropriate model data (118) and/or to ensure the consistent application of the appropriate system rules. Thus, the knowledge maintenance engine 134 could be used to select a motion model 122 (e.g., a motion model appropriate for the lung in the exemplary lung cancer application), anatomic 120 and physiology 124 models (e.g., an anatomic model for the lung for FDG-PET images), and a disease model 126 (e.g., a disease model for lung cancer). Note that the knowledge maintenance engine 134 may also operate at various points during the process as desired, for example to ensure that the selected models are consistent with those selected previously in the case or those used in similar cases. The various imaging processing components may also directly access a rule base or set which is used to carry out some or all of the functions of the knowledge maintenance engine 134.

At 204, the lesion detector 108 analyzes the image data 106 generated by the baseline scan to detect the presence of one or more tumors.

The registration processor 110 registers the images at step 206. In the exemplary case of a combined PET/CT imaging examination, the registration processor would ordinarily be used to compensate for patient and/or organ motion occurring between the PET and CT portions of, or otherwise during, the image acquisition.

At 208, the lesion quantifier 114 operates in conjunction with the physiology model(s) 124 to generate normalized lesion functional data. In the case of an FDG-PET examination, for example, the lesion quantifier 114 calculates baseline calibrated SUVs which characterize the initial activity of the various lesions. Note that, depending on patient, protocol, disease, or other application specific requirements, calibrated data indicative of additional or different lesion functional data, functional attributes, or structural attributes may also be generated.

A first follow up scan is obtained at step 210, typically after one or more courses of chemotherapy, external radiotherapy, or other desired treatment. The CT scan may be of less than diagnostic quality, for example using a relatively lower dose which generates image data 106 of sufficient quality of image registration purposes. Note also that the protocol of the follow up functional image acquisition may be different from that of the baseline acquisition, whether intentionally or otherwise. As one example, the time between the administration of the FDG or other tracer and the functional imaging examination may vary due to equipment or technician availability, differences in patient preparation time, or other factors. Variations in patient physiology may also come into play. For example, a diabetic patient may exhibit different insulin levels at the time of the initial and the follow up scans.

At 212, the lesion detector 108 analyzes the image data 106 from the follow up scan to detect the presence of lesion(s).

At 214, the lesion tracker 112 identifies the correspondence between the lesions identified in the baseline and follow up image, either alone or in combination with information from the registration processor 110.

At 216, the lesion quantifier 114 generates calibrated or normalized lesion functional data for the lesion(s) identified in the follow up scan. Thus, in the exemplary case in which the time between the administration of the relevant tracer and the functional imaging examination is different, or in the exemplary case of a diabetic patient, the information from the physiology model(s) 124 serves to correct for or otherwise reduce the impact of the variations.

At 218, the trend analyzer 116 analyzes the calibrated functional data to evaluate the response of the lesion(s) to the applied therapy, with the responses again being stored in a suitable memory. Again in the exemplary case of an FDG PET image acquisition, trend analyzer may consider among other factors changes in the calibrated SUVs of the various lesions. Note that the responses of the various identified lesions may be analyzed and evaluated separately so that the responses of various lesions may be considered individually.

At 220, the information from the trend analyzer 116 is used to predict the response to a proposed course of treatment. Alternative treatments may also be proposed.

As indicated generally at 222, 224, 226, 228 one or more additional follow up scans may be obtained and treatments applied as desired. Again in the exemplary oncologic application, follow up scans may be obtained after each of a number of chemotherapy cycles.

Note that the order of the foregoing steps and hence the functional relationship between the various system components may be varied under the control of the knowledge maintenance engine 134 or otherwise. In one such example, the registration processor 110 may be applied prior to the lesion detector 110 so that the image registration is performed prior the lesion detection operation. In another, the lesion quantification may be performed relatively earlier in the process, for example prior to the registration of the various images of the time series of images or the operation of the lesion tracker 112.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An apparatus comprising:
a lesion detector which detects lesions in medial image data from an imaging examination of a patient;
a lesion quantifier in operative communication with the lesion detector, wherein the lesion quantifier uses first functional image data from a first imaging examination of the patient conducted before the application of a therapy to the patient to generate first lesion functional data for a first detected lesion, and wherein the lesion quantifier uses second functional image data from a second imaging examination of the patient conducted after the application the therapy to generate second lesion functional data for the first detected lesion;
a trend analyzer which identifies a difference between the first and second lesion functional data.

2. The apparatus of claim 1 further including a lesion tracker which determines a correspondence between a lesion detected in image data from the first imaging examination and a lesion detected in image data from the second imaging examination.

3. The apparatus of claim 2 including a motion model, wherein the lesion tracker uses the motion model to determine the correspondence.

4. (canceled)

5. The apparatus of claim 1 further including a physiology model, wherein the lesion quantifier uses the physiology model to model an expected behavior of a tracer applied to the patient in connection with the first functional medical imaging examination.

6. (canceled)

7. The apparatus of claim 1 wherein the first imaging examination is conducted according to a first protocol and the second imaging is conducted according to a second protocol and the lesion quantifier applies a correction which reduces an effect of a variation between the first and second protocols.

8. The apparatus of claim 1 wherein the lesion quantifier applies a patient specific morphological correction or specific physiological correction.

9. (canceled)

10. The apparatus of claim 1 further including a disease model, wherein the trend analyzer uses the disease model to model a response of the first detected lesion to the applied treatment.

11. The apparatus of claim 1 further including a PET/CT scanner.

12. The apparatus of claim 1 wherein the lesion is a tumor and applied therapy includes at least one of an external radiotherapy, a chemotherapy, a brachytherapy, an ablation therapy, and a molecular therapy.

13. The apparatus of claim 1 wherein the lesion functional data includes a hypoxia, cell proliferation, or standard uptake value.

14. The apparatus of claim 1 wherein the lesion quantifier uses third functional image data generated from a third functional imaging examination of the patient conducted after the application of a second therapy to the patient to generate third lesion functional data for the first detected lesion.

15. The apparatus of claim 1 wherein the lesion quantifier uses the first functional image data to generate first lesion functional data for a second detected lesion, the lesion quantifier uses the second functional image data to generate second lesion functional data for the second detected lesion, and the trend analyzer identifies a change between the first lesion functional data for the second detected lesion and the second lesion functional data for the second detected lesion after the application of the therapy.

16. A method comprising:
calibrating, first functional data generated using data from a first imaging examination of a patient to generate first calibrated functional data indicative of a functional characteristic of a lesion;
calibrating second functional data generated using data from a second imaging examination of the patient conducted after the application of a first therapy to the patient to generate second calibrated functional data indicative of the functional characteristic of the lesion;
using the first and second calibrated functional data to evaluate a response of the lesion to the first therapy.

17. (canceled)

18. The method of claim 16 including
identifying the lesion in first medical image data from the first imaging examination;
identifying the lesion in second medical image data from the second imaging examination.

19. The method of claim 18 wherein the first medical imaging data includes first functional imaging data and first structural imaging data acquired at a first radiation dose and the second medical imaging data includes second functional imaging data and second structural imaging data acquired at second radiation dose which is lower than the first radiation dose.

20. The method of claim 18 including identifying a plurality of lesions in the first medical image data, identifying a plurality of lesions in the second medical image data, and determining a correspondence between lesions identified in the first and second medical image data.

21. The method of claim 16 wherein the method includes using the response of the lesion to the first therapy to establish a second therapy.

22. The method of claim 21 including presenting a confidence level of the second therapy to a human user.

23. The method of claim 16 wherein calibrating the first functional data includes applying a patient-specific metabolic correction.

24. The method of claim 16 wherein calibrating the second functional data includes applying a physiological model which includes at least one of a tracer uptake location, a tracer uptake time, or a tracer washout time.

25. (canceled)

26. The method of claim 16 including
identifying a plurality of lesions in the second imaging data;
determining a correspondence between lesions detected in the second image data and lesions identified in the first image data.

27. The method of claim 16 including using a motion model to model an expected motion of a detected lesion.

28. The method of claim 16 including using data from the second imaging examination to calculate lesion functional data for the detected lesion.

29. The method of claim 16 including receiving physiological model data over a computer communication network.

30. A computer readable storage medium containing instructions which, when executed out by a computer, cause the computer to carry out a method which includes:
using medical image data from a first functional medical imaging examination of a patient to generate first lesion functional data for a lesion present in the anatomy of the patient;
using the first lesion functional data and second lesion functional data for the lesion obtained from a second functional medical imaging examination of the patient to evaluate the response of the lesion to an applied therapy.

31. The computer readable storage medium of claim 30 wherein the method includes using medical image data from the second functional medical imaging examination of the patient to generate the second lesion functional data.

32. (canceled)

33. (canceled)

34. (canceled)

35. (canceled)

36. A computer readable storage medium containing a data structure which includes:
a first motion model containing data which, when accessed by a lesion tracker, describes an expected motion of a lesion detected in data from a medical imaging examination of a patent.
a first physiology model containing data which, when accessed by a lesion quantifier, describes an expected behavior of a first tracer applied to the patient in connection with a functional medical imaging examination.

37. The computer readable storage medium of claim 36, wherein the data structure includes a disease model which, when accessed by trend analyzer, describes an expected response of the lesion to an applied therapy.

38. (canceled)

39. (canceled)

40. (canceled)

41. The computer readable storage medium of claim 36, wherein the data structure includes
a second physiology model containing data which, when accessed by the lesion quantifier, describes an expected behavior of a second tracer applied to a patient in connection with a functional medical imaging examination.

42. (canceled)

43. (canceled)

44. A method for use in computer assisted therapy monitoring, the method comprising:
using information which describes an image protocol used in connection with a functional imaging examination of a patient to select model data;
using the selected model data to vary an operation of a component of a computer assisted therapy monitoring system.

45. The method of claim 44 wherein the model data includes a physiology model and the component includes a lesion quantifier.

46. (canceled)

47. (canceled)

48. The method of claim 44 wherein the image protocol includes at least one of an imaging modality and a tracer.

49. (canceled)
